(19) United States
(12) Patent Application Publication
(10) Pub. No.: US 2025/0204994 A1
(43) Pub. Date: Jun. 26, 2025

(54) Surgical Accessory Element-Based Setup of a Robotic System

(71) Applicant: Intuitive Surgical Operations, Inc., Sunnyvale, CA (US)

(72) Inventors: Aidean Sharghi Karganroodi, Pearland, TX (US); Omid Mohareri, San Francisco, CA (US); Dinesh Rabindran, San Jose, CA (US)

(21) Appl. No.: 18/851,598

(22) PCT Filed: Mar. 27, 2023
(86) PCT No.: PCT/US2023/016424

§ 371 Date: Sep. 26, 2024

Related U.S. Application Data
(60) Provisional application No. 63/324,870, filed on Mar. 29, 2022.

(51) Int. Cl. A61B 34/30; A61B 34/20; A61B 90/00; A61B 90/90; G06V 20/50; G16H 40/20; G16H 40/63

(52) U.S. Cl. CPC A61B 34/30; A61B 90/90; G06V 20/50; G16H 40/20; G16H 40/63; A61B 2034/2055; A61B 2034/2065; A61B 2090/0805; A61B 2090/373; A61B 2090/3937; G06V 2201/034

(57) ABSTRACT

An illustrative setup system may be configured to identify, based on data representative of a scene as captured by one or more sensors, a group of surgical accessory elements within the scene, and perform, based on information representative of one or more positions of the group of surgical accessory elements within the scene, an operation for setup of one or more components of a robotic system relative to the group of surgical accessory elements.

### RELATED APPLICATIONS

The present application claims priority to U.S. Provisional Patent Application No. 63/324,870, filed Mar. 29, 2022, the contents of which is hereby incorporated by reference in its entirety.

### BACKGROUND INFORMATION

A robotic system may be used to perform various types of medical procedures. The robotic system may typically be setup (e.g., navigated and/or docked) relative to a patient in preparation for the medical procedure to be performed. The setup of the robotic system relative to the patient may depend on a number of factors such as the type of medical procedure, the patient's physical characteristics, the site layout, and/or preferences of medical personnel (e.g., medical personnel performing the setup of the robotic system). In some scenarios, setup of the robotic system may be difficult and/or time consuming, particularly for relatively less-experienced personnel.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system includes a memory storing instructions and a processor communicatively coupled to the memory and configured to execute the instructions to: identify, based on data representative of a scene as captured by one or more sensors, a group of surgical accessory elements within the scene; and perform, based on information representative of one or more positions of the group of surgical accessory elements within the scene, an operation for setup of one or more components of a robotic system relative to the group of surgical accessory elements.

An illustrative system includes a robotic system; one or more sensors configured to capture a scene; and a control system communicatively coupled with the robotic system and the one or more sensors. The control system may be configured to: identify, based on data representative of the scene as captured by the one or more sensors, a group of surgical accessory elements within the scene; and perform, based on information representative of one or more positions of the group of surgical accessory elements within the scene, an operation for setup of the robotic system relative to the group of surgical accessory elements.

An illustrative method includes identifying, by a setup system and based on data representative of a scene as captured by one or more sensors, a group of surgical accessory elements within the scene; and performing, by a setup system and based on information representative of one or more positions of the group of surgical accessory elements within the scene, an operation for setup of one or more components of a robotic system relative to the group of surgical accessory elements.

An illustrative non-transitory computer-readable medium may store instructions that, when executed, direct a processor of a computing device to: identify, based on data representative of a scene as captured by one or more sensors, a group of surgical accessory elements within the scene; and perform, based on information representative of one or more positions of the group of surgical accessory elements within the scene, an operation for setup of one or more components of a robotic system relative to the group of surgical accessory elements.

An illustrative computer-assisted medical system includes a robotic system having one or more repositionable manipulator arms; one or more sensors configured to capture a scene depicting a group of surgical accessory elements within the scene; and a computing device communicatively coupled to the robotic system and the one or more sensors. The computing device may be configured to: identify, based on data representative of the scene as captured by the one or more sensors, the group of surgical accessory elements within the scene; and perform, based on information representative of one or more positions of the group of surgical accessory elements within the scene, an operation for setup of the one or more repositionable manipulator arms of the robotic system relative to the group of surgical accessory elements.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 depicts an illustrative implementation of a robotic system according to principles described herein.

FIG. 2 depicts an illustrative implementation of a setup system according to principles described herein.

FIG. 3 depicts an illustrative method of operating a setup system according to principles described herein.

FIG. 4 depicts an illustrative implementation of a plurality of sensors according to principles described herein.

FIG. 5A depicts an illustrative implementation of a group of surgical accessory elements according to principles described herein.

FIG. 5B depicts an enlarged side view of the group of surgical accessory elements of FIG. 5A encircled by circle A of FIG. 5A.

FIG. 5C depicts an enlarged top plan view of the group of surgical accessory elements of FIG. 5A encircled by circle A of FIG. 5A.

FIG. 6 depicts an illustrative implementation of positioning a robotic system relative to a group of surgical accessory elements according to principles described herein.

FIG. 7 depicts an illustrative implementation of a user interface according to principles described herein.

FIG. 7A depicts an illustrative implementation of a display of the user interface of FIG. 7.

FIG. 8 depicts another illustrative method of operating a setup system according to principles described herein.

FIG. 9 shows an illustrative computing system according to principles described herein.

### DETAILED DESCRIPTION

An illustrative setup system may be configured to perform one or more operations to assist with setup of one or more components of a robotic system relative to a group of surgical accessory elements (e.g., in preparation for performance of a medical procedure).

In certain implementations, for example, the setup system may be configured to identify, based on data representative of a scene as captured by one or more sensors, a group of surgical accessory elements within the scene. The setup system may further be configured to perform, based on information representative of one or more positions of the group of surgical accessory elements within the scene, an operation for setup of one or more components of a robotic system relative to the group of surgical accessory elements. In some implementations, the operation may involve positioning one or more components of the robotic system relative to the group of surgical accessory elements and/or providing instructions for positioning one or more components of the robotic system relative to the group of surgical accessory elements.

To illustrate, the setup system may receive data representative of the scene captured by the one or more sensors. The scene may include a three-dimensional (3D) scene that can be represented by 3D data or two dimensional (2D) data. The setup system may identify, based on the received data, the group of surgical accessory elements within the scene. In some implementations, the group of surgical accessory elements may comprise one or more markers (e.g., a retroreflective marker) that may be detected by the one or more sensors for identifying the group of surgical accessory elements within the scene. The setup system may further determine information representative of one or more positions of the group of surgical accessory elements within the scene. In some implementations, the one or more positions may include one or more 3D positions that are determined such as by generating depth data representative of one or more distances of one or more components of the robotic system relative to the group of surgical accessory elements.

The setup system may, based on the information representative of the one or more positions of the group of surgical accessory elements, direct movement of one or more components of the robotic system relative to the group of surgical accessory elements. For example, based on the generated depth data, the setup system may cause the one or more components of the robotic system to move and/or the setup system may provide instructions for a user of the robotic system to move the one or more components of the robotic system. As the robotic system moves, the setup system may update the generated depth data over time. In some implementations, the setup system may continue to direct movement of the one or more components of the robotic system until the robotic system reaches a desired position relative to the group of surgical accessory elements.

The principles described herein may result in improved robotic system setup compared to conventional techniques that are not based on an identified group of surgical accessory elements, as well as provide other benefits as described herein. For example, setup of one or more components of a robotic system based on an identified group of surgical accessory elements may allow the robotic system to be more quickly and/or easily positioned relative to a group of surgical accessory elements in preparation for performance of a medical procedure. Such setup of the robotic system may also allow the robotic system to be moved into a more optimal and/or accurate position relative to the group of surgical accessory elements.

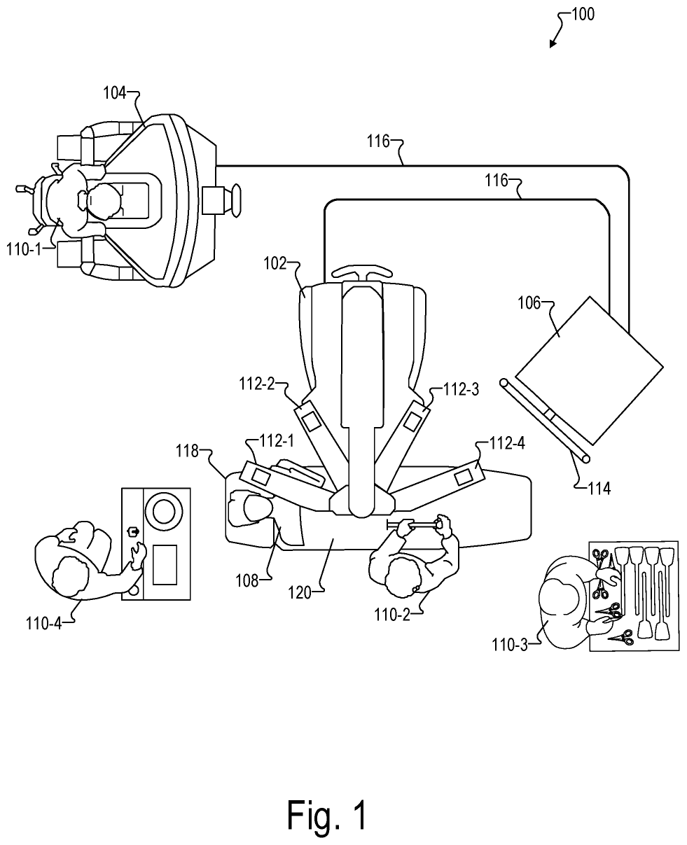

FIG. 1 shows an illustrative robotic system 100 that may be used to perform various types of medical procedures including surgical and/or non-surgical procedures.

As shown, robotic system 100 may include a manipulator assembly 102 (a manipulator cart is shown in FIG. 1), a user control apparatus 104, and an auxiliary apparatus 106, all of which are communicatively coupled to each other. Robotic system 100 may be utilized by a medical team to perform a computer-assisted medical procedure or other similar operation on a body of a patient 108 or on any other body as may serve a particular implementation. In some implementations, the body of patient 108 may be positioned on an operating room table 118 and/or covered by a drape 120. As shown, the medical team may include a first user 110-1 (such as a surgeon for a surgical procedure), a second user 110-2 (such as a patient-side assistant), a third user 110-3 (such as another assistant, a nurse, a trainee, etc.), and a fourth user 110-4 (such as an anesthesiologist for a surgical procedure), all of whom may be collectively referred to as users 110, and each of whom may control, interact with, or otherwise be a user of robotic system 100. More, fewer, or alternative users may be present during a medical procedure as may serve a particular implementation. For example, team composition for different medical procedures, or for non-medical procedures, may differ and include users with different roles.

While FIG. 1 illustrates an ongoing minimally invasive medical procedure such as a minimally invasive surgical procedure, it will be understood that robotic system 100 may similarly be used to perform open medical procedures or other types of operations.

For example, operations such as exploratory imaging operations, mock medical procedures used for training purposes, and/or other operations may also be performed.

As shown in FIG. 1, manipulator assembly 102 may include one or more manipulator arms 112 (e.g., manipulator arms 112-1 through 112-4) to which one or more instruments may be coupled. In some implementations, manipulator assembly 102 may be positioned proximate to a patient 108 (e.g., as a patient side cart) for the performance of a medical procedure. For example, the instruments may be used for a computer-assisted medical procedure on patient 108 (e.g., in a surgical example, by being at least partially inserted into patient 108 and manipulated within patient 108). While manipulator assembly 102 is depicted and described herein as including four manipulator arms 112, it will be recognized that manipulator assembly 102 may include a single manipulator arm 112 or any other number of manipulator arms as may serve a particular implementation. While the example of FIG. 1 illustrates manipulator arms 112 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 112 shown in FIG. 1. In some implementations, manipulator assembly 102 may be considered a robotic system that is a component of robotic system 100.

During the medical operation, user control apparatus 104 may be configured to facilitate teleoperational control by user 110-1 of manipulator arms 112 and instruments attached to manipulator arms 112. To this end, user control apparatus 104 may provide user 110-1 with imagery of an operational area associated with patient 108 as captured by an imaging device. To facilitate control of instruments, user control apparatus 104 may include a set of master controls. These master controls may be manipulated by user 110-1 to control movement of the manipulator arms 112 or any instruments coupled to manipulator arms 112.

Auxiliary apparatus 106 may include one or more computing devices configured to perform auxiliary functions in support of the medical procedure, such as providing insufflation, electrocautery energy, illumination or other energy for imaging devices, image processing, or coordinating components of robotic system 100. In some examples, auxiliary apparatus 106 may be configured with a display monitor 114 configured to display one or more user interfaces, or graphical or textual information in support of the medical procedure. In some instances, display monitor 114 may be implemented by a touchscreen display and provide user input functionality. Augmented content provided by a region-based augmentation system may be similar, or differ from, content associated with display monitor 114 or one or more display devices in the operation area (not shown).

Manipulator assembly 102, user control apparatus 104, and auxiliary apparatus 106 may be communicatively coupled one to another in any suitable manner. For example, as shown in FIG. 1, manipulator assembly 102, user control apparatus 104, and auxiliary apparatus 106 may be communicatively coupled by way of control lines 116, which may represent any wired or wireless communication link as may serve a particular implementation. To this end, manipulator assembly 102, user control apparatus 104, and auxiliary apparatus 106 may each include one or more wired or wireless communication interfaces, such as one or more local area network interfaces, Wi-Fi network interfaces, cellular interfaces, and so forth.

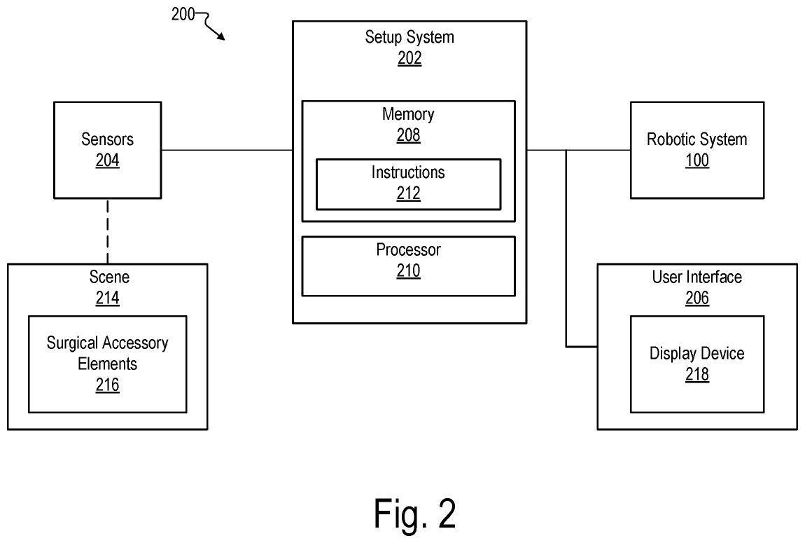

FIG. 2 shows an illustrative implementation 200 configured to assist with setup of one or more components of robotic system 100 (e.g., manipulator assembly 102 and/or manipulator arms 112). As shown, implementation 200 includes a setup system 202 communicatively coupled (e.g., wired and/or wirelessly) with robotic system 100, a plurality of sensors 204, and a user interface 206. Implementation 200 may include additional or alternative components as may serve a particular implementation. In some examples, components of setup system 202, sensors 204, and/or user interface 206 may be implemented by a computer-assisted medical system, such as robotic system 100 described above.

Setup system 202 may be implemented by one or more computing devices and/or computer resources (e.g., processors, memory devices, storage devices, etc.) as may serve a particular implementation. As shown, setup system 202 may include, without limitation, a memory 208 and a processor 210 selectively and communicatively coupled to one another. Memory 208 and processor 210 may each include or be implemented by computer hardware that is configured to store and/or process computer software. Various other components of computer hardware and/or software not explicitly shown in FIG. 2 may also be included within setup system 202. In some examples, memory 208 and/or processor 210 may be distributed between multiple devices and/or multiple locations as may serve a particular implementation.

Memory 208 may store and/or otherwise maintain executable data used by processor 210 to perform any of the functionality described herein. For example, memory 208 may store instructions 212 that may be executed by processor 210. Memory 208 may be implemented by one or more memory or storage devices, including any memory or storage devices described herein, that are configured to store data in a transitory or non-transitory manner. Instructions 212 may be executed by processor 210 to cause setup system 202 to perform any of the functionality described herein. Instructions 212 may be implemented by any suitable application, software, code, and/or other executable data instance. Additionally, memory 208 may also maintain any other data accessed, managed, used, and/or transmitted by processor 210 in a particular implementation.

Processor 210 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 210 (e.g., when processor 210 is directed to perform operations represented by instructions 212 stored in memory 208), setup system 202 may perform various operations as described herein.

Sensors 204 may include any sensor(s) or imaging device(s) configured to capture a scene 214 (e.g., a 3D scene or a 2D scene). For example, sensors 204 may include video imaging devices, infrared imaging devices, visible light imaging devices, non-visible light imaging devices, intensity imaging devices (e.g., color, grayscale, black and white imaging devices), depth imaging devices (e.g., stereoscopic imaging devices, time-of-flight imaging devices, infrared imaging devices, red-green-blue (RGB) imaging devices, red-green-blue and depth (RGB-D) imaging devices, light detection and ranging (LIDAR) imaging devices, etc.), any other imaging devices, or any combination or sub-combination of such imaging devices.

Sensors 204 may be positioned relative to scene 214 and may be configured to image scene 214 by concurrently capturing images of scene 214. As used herein, an “image” may include a video stream and/or one or more still image snapshots. Sensors 204 may be configured to capture images of scene 214 at any suitable capture rates. Sensors 204 may be synchronized in any suitable way for synchronous capture of images of scene 214. The synchronization may include operations of sensors 204 being synchronized and/or data sets output by sensors 204 being synchronized by matching data sets to common points in time.

Scene 214 may include any environment and/or elements of an environment that may be imaged by sensors 204. For example, scene 214 may include a tangible real-world scene of physical elements. In certain illustrative examples, scene 214 is associated with a medical procedure such as a surgical procedure. For example, scene 214 may include a surgical scene at a surgical site such as a surgical facility, operation room, or the like. For instance, scene 214 may include all or part of an operating room in which a surgical procedure may be performed on a patient. In certain implementations, scene 214 includes an area of an operating room proximate to a robotic system (e.g., robotic system 100) that is used to perform a surgical procedure. While certain illustrative examples described herein are directed to scene 214 including a scene at a surgical facility, one or more principles described herein may be applied to other suitable scenes in other implementations.

In the illustrated implementation, a group of surgical accessory elements 216 is included within scene 214 (e.g., positioned within a field of view of sensors 204). Surgical accessory elements 216 may be positioned on or near patient 108 or other subject such as to assist with performance of a medical procedure. To illustrate, surgical accessory elements 216 may be positioned, at least partially, outside of patient 108. In some implementations, the surgical accessory elements may include access elements that are configured to extend outward from patient 108 such as to provide access to an interior portion of patient 108. For example, access elements may include, without limitation, one or more of: a cannula, an access port, an obturator, or a trocar. Still other suitable surgical accessory elements may be used. For example, the surgical accessory elements may include an operating room table 118 on which patient 108 may be positioned and/or a drape 120 that may be configured to cover at least a portion of patient 108.

User interface 206 of the illustrated implementation comprises a display device 218. Display device 218 may be implemented by a monitor or other suitable device configured to display information to a user (e.g., users 110). For example, display device 218 may be configured to display an image or other information based on 3D scene 214 captured by sensors 204. In some implementations, user interface 206 may further include any suitable device (e.g., a button, joystick, touchscreen, keyboard, handle, etc.) configured to receive a user input (e.g., from users 110) such as to control or direct one or more components of robotic system 100.

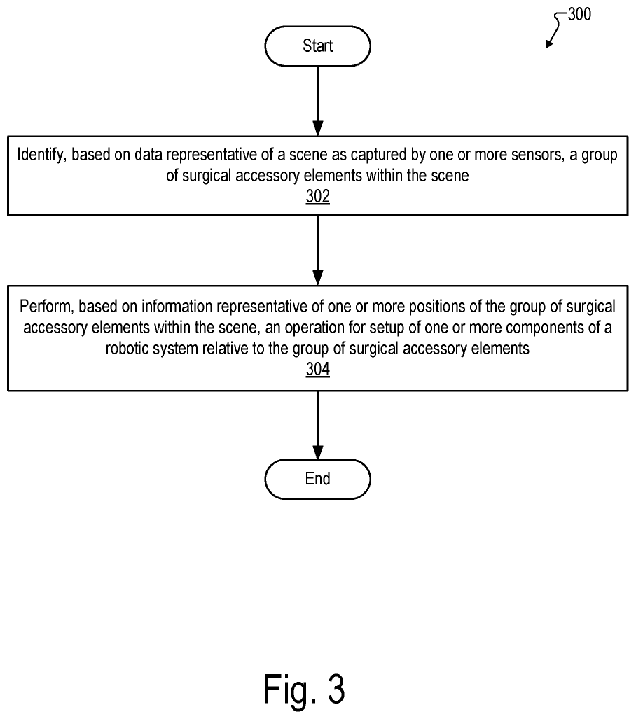

FIG. 3 shows an illustrative method 300 that may be performed by setup system 202. While FIG. 3 illustrates exemplary operations according to one embodiment, other embodiments may omit, add to, reorder, and/or modify any of the operations shown in FIG. 3. Moreover, each of the operations depicted in FIG. 3 may be performed in any of the ways described herein.

As shown, setup system 202 may, at operation 302, identify, based on data representative of scene 214 as captured by the one or more sensors 204, the group of surgical accessory elements 216 within scene 214. Setup system 202 may, at operation 304, perform, based on information representative of one or more positions of the group of surgical accessory elements 216 within scene 214, an operation for setup of one or more components of robotic system 100 relative to the group of surgical accessory elements 216.

The operation for setup of one or more components of robotic system 100 may include positioning one or more components of robotic system 100 (e.g., manipulator assembly 102 and/or manipulator arms 112) relative to the group of surgical accessory elements 216. For example, setup system 202 may cause movement of the one or more components of robotic system 100 and/or provide instructions to a user for moving the one or more components of robotic system 100. During positioning of the one or more components of robotic system 100, the one or more components may be aligned with the group of surgical accessory elements 216, the one or more components may be aligned with an individual surgical accessory element of the group of surgical accessory elements 216, and/or the one or more components may be aligned with a position that is determined based on positions and/or orientations of the group of surgical accessory elements 216.

Still other operations may be performed by setup system 202 for setup of one or more components of robotic system 100. For example, the operation may additionally or alternatively include identifying a type of medical procedure to be performed using robotic system 100 and using the identified type of medical procedure to cause movement of the one or more components of robotic system 100 and/or provide instructions to a user for moving the one or more components of robotic system 100. In some implementations, the operation for setup may be performed based on identifying one surgical accessory element 216. For example, one surgical accessory element 216 may provide sufficient information for positioning one or more components of robotic system 100. Additionally or alternatively, a first operation for setup may be performed based on a single surgical accessory element 216 and a second operation for setup may be performed based on the group of surgical accessory elements 216.

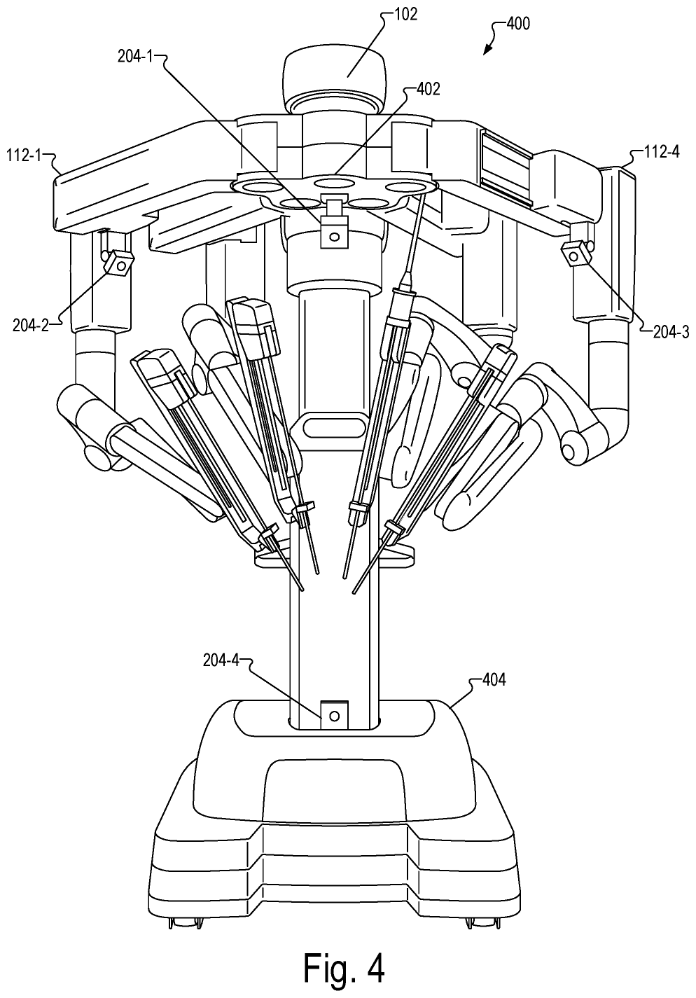

FIG. 4 depicts an illustrative implementation 400 of sensors 204 (e.g., sensors 204-1 through 204-4) for capturing data representative of scene 214. As shown, sensors 204 are attached to components of manipulator assembly 102 of robotic system 100. For example, sensor 204-1 may be attached to an orienting platform (OP) 402 of manipulator assembly 102, sensor 204-2 may be attached to manipulator arm 112-1 of manipulator assembly 102, sensor 204-3 may be attached to manipulator arm 112-4 of manipulator assembly 102, and sensor 204-4 may be attached to a base 404 of manipulator assembly 102. In implementations in which manipulator assembly 102 is positioned proximate to a patient (e.g., as a patient side cart) and/or relative to the group of surgical accessory elements 216, placement of sensors 204 at strategic locations on manipulator assembly 102 may provide advantageous imaging viewpoints proximate to a patient, the group of surgical accessory elements 216, and/or a surgical procedure performed on the patient.

The illustrated placements of sensors 204 to components of manipulator assembly 102 are illustrative. Additional and/or alternative placements of any suitable number of sensors 204 on manipulator assembly 102, other components of robotic system 100, and/or other components at a surgical facility may be used in other implementations. Sensors 204 may be attached to components of manipulator assembly 102, other components of robotic system 100, and/or other components at a surgical facility in any suitable way. Sensors 204 may be mounted on a fixed component and/or sensors 204 may be mounted on a moveable component such that sensors 204 may be moveable relative to scene 214 and/or relative to one or more other sensors 204. A registration process may be performed (e.g., by setup system 202) to register sensors 204 to setup system 202. The registration process may be configured to determine spatial relationships between sensors 204 (e.g., viewpoints of sensors 204) and components of robotic system 100. This may result in known or deterministic relationships of component tracking data, such as robotic kinematics data of robotic system 100 (e.g., data descriptive of velocity and acceleration of robotic system 100), to sensors 204. The determined spatial relationships may be used to determine spatial information for sensors 204 (e.g., positions, orientations, poses, viewpoints, and/or fields of view of the sensors 204 based on spatial information for the components of robotic system 100).

Sensors 204 may be configured to generate and/or output data representative of scene 214. The data may include, without limitation, one or more of an image, color, grayscale, saturation, intensity, or brightness captured by sensors 204. The data may, in some instances, be associated with data points expressed in a common coordinate frame such as 3D voxels or two-dimensional (2D) pixels of images captured by sensors 204.

In some implementations, setup system 202 may be configured to fuse data points associated with images captured at a common point in time from different sensors 204 having different viewpoints of scene 214. In certain examples, the fusing may include merging aligned (or overlapping) voxels or pixels, such as by blending intensity and/or depth values for aligned voxels or pixels. The blending may include weighted blending in which the data points being blended are weighted based on one or more factors, such as which of sensors 204 has the best view of a data point (e.g., by more heavily weighting data captured by the sensor 204 with the best viewing angle). In certain examples, the fusing may additionally or alternatively include stitching non-overlapping voxels or pixels together, such as by stitching images together along non-overlapping boundaries of the images.

Setup system 202 may be configured to receive the data representative of scene 214 from sensors 204 and to identify, based on the received data, the group of surgical accessory elements 216 within scene 214.

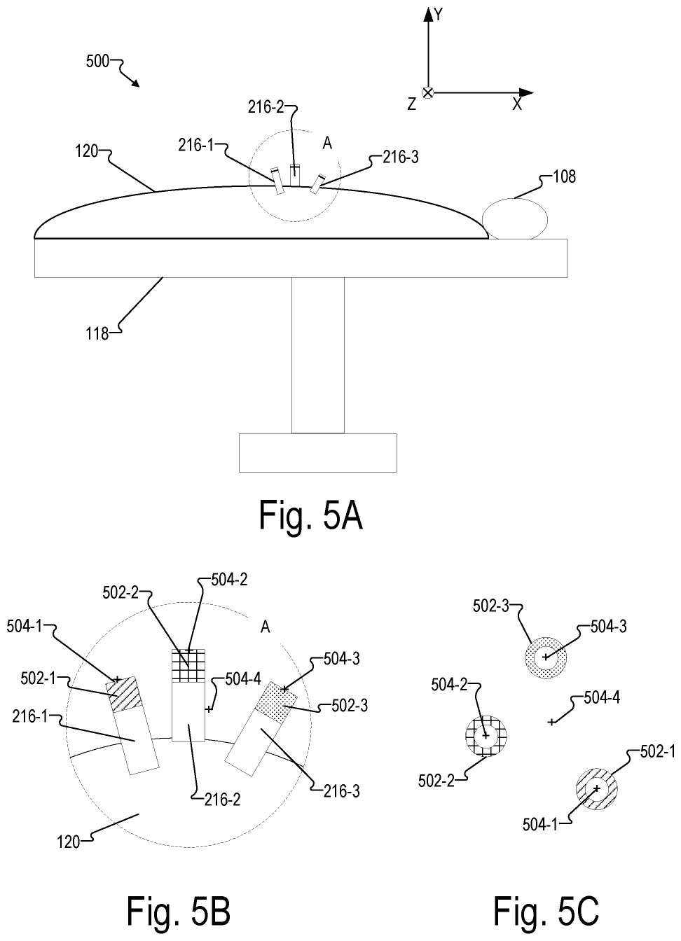

In some implementations, the group of surgical accessory elements 216 may comprise one or more markers and setup system 202 may be configured to detect the one or more markers in scene 214. As an illustrative example, FIGS. 5A-5C show an implementation 500 of a group of surgical accessory elements 216 (e.g., elements 216-1 through 216-3) positioned on a patient 108. As shown, each surgical accessory element 216 comprises a marker 502 (e.g., markers 502-1 through 502-3). Each marker 502 may include a retroreflective material (e.g., a retroreflective sheeting, tape, paint, ink, etc.) configured to reflect light. These surgical accessory elements 216 and markers 502 are merely illustrative, such that any suitable number of markers 502 may be associated with any suitable number of surgical accessory elements 216.

In instances where the group of surgical accessory elements 216 comprise one or more retroreflective markers 502, sensors 204 (e.g., depth imaging devices) may comprise a light source configured to emit light and an image sensor configured to detect a reflected part of the light. For example, the light source of sensors 204 may emit light toward the group of surgical accessory elements 216 within scene 214, the one or more markers 502 of the group of surgical accessory elements 216 may reflect light back to the sensors 204, and the image sensor of sensors 204 may detect the reflected light when the one or more markers 502 are located within a field of view of the image sensor.

The reflected light may form bright locations (e.g., one or more locations within the received data having a brightness greater than other locations within the received data and/or having a brightness above a threshold) at the locations of the one or more markers 502 within the received data from sensors 204. Setup system 202 may detect and cluster concentrations of bright locations within the received data from sensors 204 to detect the one or more markers 502 and identify the group of surgical accessory elements 216 within scene 214. In some implementations, setup system 202 may be configured to filter the received data from sensors 204 (e.g., to remove bright locations having a brightness below a threshold). Additionally or alternatively, setup system 202 may be configured to remove outlier bright locations that may be spaced away from other bright locations.

In some scenarios, setup system 202 may be configured to identify one or more surgical accessory elements 216 relative to another surgical accessory element 216 (e.g., setup system 202 may identify a type of surgical accessory element 216 relative to another surgical accessory element 216, such as a cannula, an access port, a robotic port, an obturator, a trocar, etc.). For example, the group of surgical accessory elements 216 may include a first surgical accessory element (e.g., 216-1) comprising a first retroreflective marker (e.g., 502-1) and a second surgical accessory element (e.g., 216-2 and/or 216-3) comprising a second retroreflective marker (e.g., 502-2 and/or 502-3) different from the first retroreflective marker. In the illustrative implementation, the first retroreflective marker (e.g., 502-1) has a different pattern than the second retroreflective marker (e.g., 502-2 and/or 502-3), though other suitable differences may be used. For example, surgical accessory elements 216 may include a different number of markers 502, a different size of markers 502, a different spatial arrangement of markers 502, a different shape of markers 502, and/or a different reflective characteristic of markers 502.

Additionally or alternatively, setup system 202 may be configured to identify the group of surgical accessory elements 216 within scene 214 by implementing and applying artificial intelligence algorithms such as machine learning algorithms. Any suitable form of artificial intelligence and/or machine learning may be used, including, for example, deep learning, neural networks, etc. For example, a machine learning algorithm may be generated through machine learning procedures and applied to identification operations. In some implementations, the machine learning algorithm may be directed to identifying a surgical accessory element 216 and/or a marker 502 of a surgical accessory element 216 within scene 214. The machine learning algorithm may operate as an identification function that is applied to individual and/or fused imagery to classify surgical accessory elements 216 in the imagery.

Still other suitable methods may be used for identifying the group of surgical accessory elements 216 within scene 214 in addition to or instead of machine learning algorithms. For example, setup system 202 may be configured to identify the group of surgical accessory elements 216 within scene 214 by implementing and applying object recognition algorithms. For example, an object recognition algorithm may be used to identify objects (e.g., surgical accessory elements 216) of predetermined types within the data received from sensors 204, such as by comparing the data received from sensors 204 to model object data of predetermined types of objects. Such model object data may be stored within a model database that may be communicatively coupled with setup system 202.

Once the group of surgical accessory elements 216 have been identified, setup system 202 may determine information representative of one or more positions of the identified surgical accessory elements 216 within scene 214. The determination of information representative of one or more positions of the identified surgical accessory elements 216 may include determining information representative of a 3D position of each surgical accessory element within the group of surgical accessory elements 216, a 3D position of a select one or more surgical accessory elements within the group of surgical accessory elements 216, and/or a 3D position of a location offset from the group of surgical accessory elements 216 and based on the group of surgical accessory elements 216 (e.g., a centroid of the group of surgical accessory elements 216 and/or other position relative to the group of surgical accessory elements 216). In some implementations, the information may include one or more 3D positions of the group of surgical accessory elements 216 relative to an x-axis, y-axis, and z-axis within scene 214. For example, the data captured by sensors 204 may include information representative of one or more 3D positions of the group of surgical accessory elements 216 within scene 214 (e.g., the data captured by sensors 204 may be associated with data points such as 3D voxels expressed in a 3D common coordinate frame and/or 2D pixels expressed in a 2D common coordinate frame).

In some implementations, the information representative of the one or more positions of the group of surgical accessory elements may include one or more target locations (e.g., a desired location for positioning one or more components of robotic system 100). As an illustrative example, FIGS. 5B-5C show 3D positions of target locations 504 (e.g., locations 504-1 through 504-4) associated with the group of surgical accessory elements 216. As shown, the one or more target locations 504 may include, without limitation, a location at one or more individual surgical accessory elements 216 (e.g., locations 504-1, 504-2, and/or 504-3), a centroid of the surgical accessory elements 216 (e.g., location 504-4), an individual marker 502, and/or another location associated with the group of surgical accessory elements 216.

In some implementations, the information representative of one or more positions of surgical accessory elements 216 may further include a pose (e.g., a position and orientation) of one or more surgical accessory elements 216. For example, the pose may include a degree of freedom of the one or more surgical accessory elements 216 along the x-axis, y-axis, and z-axis within scene 214. The pose may, in some instances, assist in identifying the group of the surgical accessory elements 216.

Setup system 202 may further be configured to generate, based on the information representative of the one or more positions of the group of surgical accessory elements 216, depth data representative of one or more distances of the group of surgical accessory elements 216 relative to one or more components of robotic system 100. For example, setup system 202 may be configured to determine a distance from the one or more components of robotic system 100 to be positioned relative to the one or more target locations 504.

In instances where the group of surgical accessory elements 216 include one or more retroreflective markers 502, setup system 202 may be configured to generate depth data based on a phase shift or an amount of time between the emitted light from the light source of sensors 204 and the reflected light detected by the image sensor of sensors 204. Additionally or alternatively, setup system 202 may determine the depth data by processing stereoscopic images captured by sensors 204. Setup system 202 may be configured to update the depth data over time as one or more components of robotic system 100 is moved relative to the group of surgical accessory elements 216. For example, a simultaneous localization and mapping (SLAM) algorithm may be used to construct and/or update a map of scene 214 while simultaneously keeping track of the location of the group of surgical accessory elements 216 and/or target locations within it.

Setup system 202 may further be configured to perform, based on the information representative of one or more positions of the group of surgical accessory elements 216 within the scene 214, an operation for setup of one or more components of robotic system 100.

In some implementations, the operation for setup of one or more components of robotic system 100 may include positioning manipulator assembly 102 relative to the group of surgical accessory elements 216. To illustrate, the 3D position of a target location that is offset and/or proximate to operating room table 118 may be used for moving the entire manipulator assembly 102 relative to the group of surgical accessory elements 216 (e.g., to position manipulator assembly 102 proximate to patient 108 and/or operating room table 118). Additionally or alternatively, one or more components of manipulator assembly 102 may be positioned relative to the group of surgical accessory elements 216. For example, OP 402 of manipulator assembly 102 may be raised and/or lowered relative to manipulator assembly 102 such as to position OP 402 relative to a height of operating room table 118. In some implementations, OP 402 may be extended and/or retracted relative to manipulator assembly 102 such as to collectively position manipulator arms 112 relative to a centroid (e.g., target location 504-4) of the group of surgical accessory elements 216 (e.g., OP 402 may be positioned above target location 504-4 in a direction along a y-axis of target location 504-4).

Additionally or alternatively, one or more individual manipulator arms 112 of manipulator assembly 102 may be aligned with an individual surgical accessory element of the group of surgical accessory elements 216. For example, a position of a select manipulator arm (e.g., manipulator arm 112-1) may be aligned with a position of a target location (e.g., target location 504-1) of a select surgical accessory element (e.g., surgical accessory element 216-1), such as along an x-axis, y-axis, and/or z-axis of the target location. Moreover, an orientation of the select manipulator arm may be set to correspond to an orientation and/or desired orientation of the select surgical accessory element (e.g., based on a pose of the select surgical accessory element). In some implementations, each manipulator arm may be moved individually to the desired orientation and/or a group of manipulator arms may be moved collectively to one or more desired orientations.

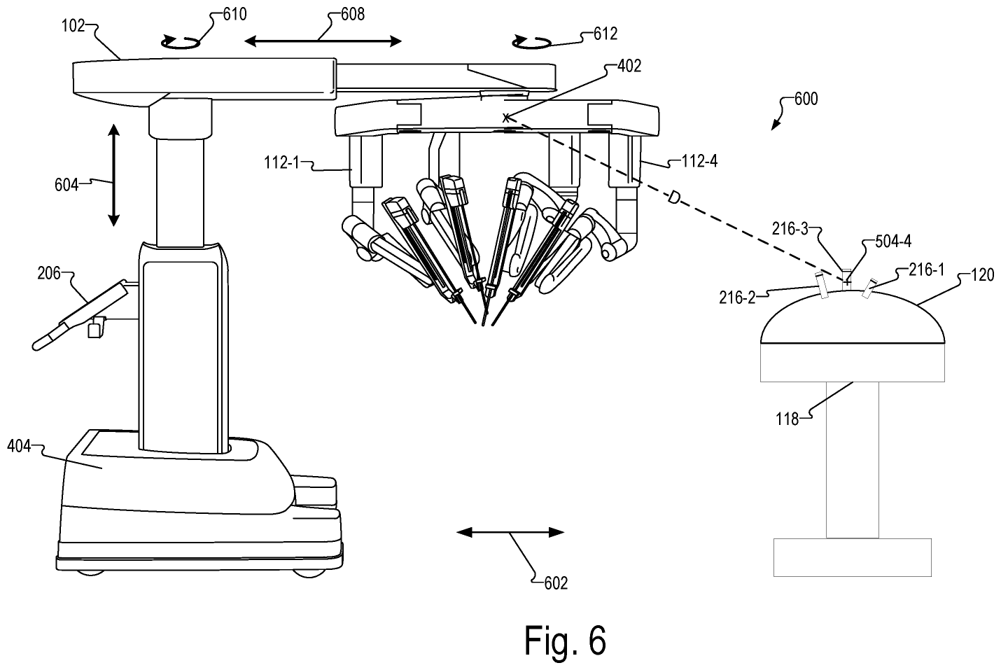

As an illustrative example, FIG. 6 shows an implementation 600 of positioning one or more components of robotic system 100 relative to the group of surgical accessory elements 216. As shown, setup system 202 may be configured to generate depth data representative of one or more distances D from one or more components of robotic system 100 (e.g., a desired one or more components of robotic system 100 to be moved) to the group of surgical accessory elements 216 (e.g., one or more target locations 504 associated with the group of surgical accessory elements 216). In the illustrated implementation, the distance D is shown from OP 402 of manipulator assembly 102 to a centroid target location 504-4 of the group of surgical accessory elements 216. This distance D is merely illustrative such that other components of robotic system 100 and/or target locations 504 of the group of surgical accessory elements 216 may be used to determine distance D.

Setup system 202 may further be configured to position the one or more components of robotic system 100 (e.g., OP 402) relative to the group of surgical accessory elements 216 (e.g., target location 504-4). For example, the entire manipulator assembly 102 may move relative to the group of surgical accessory elements 216 (e.g., along arrow 602). Manipulator arms 112 may further move collectively relative to manipulator assembly 102 and/or the group of surgical accessory elements 216. For example, OP 402 of manipulator assembly 102 may translate manipulator arms 112 (e.g., collectively extend and/or retract manipulator arms 112 along arrows 604 and/or 608) and/or rotate manipulator arms 112 (e.g., collectively rotate manipulator arms 112 along arrows 610 and/or 612). Manipulator arms 112 may further move individually relative to manipulator assembly 102 and/or the group of surgical accessory elements 216.

In some implementations, setup system 202 may be configured to cause movement of robotic system 100 such as by directing, based on kinematic data of robotic system 100, one or more components of robotic system 100 to move relative to the group of surgical accessory elements 216. For example, manipulator assembly 102, manipulator arms 112, and/or surgical instruments attached to manipulator arms 112 may include one or more displacement transducers, orientational sensors, and/or positional sensors used to generate raw (i.e., uncorrected) kinematics data (e.g., data descriptive of velocity and acceleration). Setup system 202 may be configured to use the kinematics information to track (e.g., determine poses of) and/or control the surgical instruments, as well as anything connected to the instruments and/or arms. As described herein, setup system 202 may use the kinematics information to track components of robotic system 100 (e.g., manipulator arms 112 and/or surgical instruments attached to manipulator arms 112) and/or cause movement of the components. In some implementations, setup system 202 may be communicatively coupled to one or more motors (not shown) of robotic system 100 such that setup system 202 may cause operation of the one or more motors to move the one or more components of robotic system 100.

Additionally or alternatively, setup system 202 may be configured to provide instructions to user interface 206 for positioning one or more components of robotic system 100 relative to the group of surgical accessory elements 216. The instructions may assist a user 110 with moving one or more components of robotic system 100. As shown in FIG. 6, user interface 206 may be coupled with manipulator assembly 102 such as on a rear portion of manipulator assembly 102. The illustrated placement of user interface 206 to manipulator assembly 102 is illustrative. Additional and/or alternative placements of user interface 206 on manipulator assembly 102, other components of robotic system 100, and/or other components at a surgical facility may be used in other implementations. User interface 206 may be attached to components of manipulator assembly 102, other components of robotic system 100, and/or other components at a surgical facility in any suitable way.

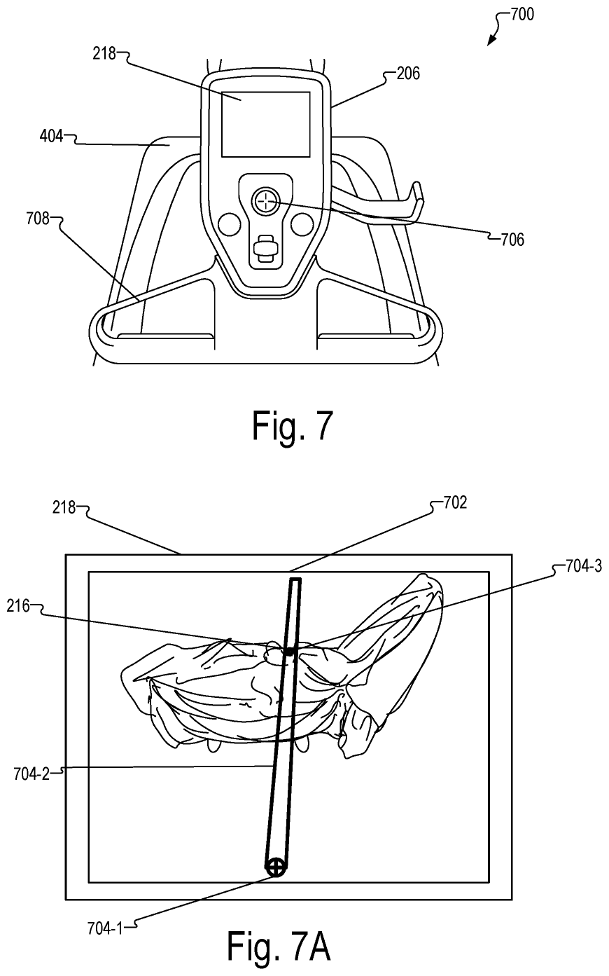

FIG. 7 shows an illustrative implementation 700 of user interface 206 comprising display device 218 configured to display the instructions. FIG. 7A shows display device 218 displaying an illustrative example of instructions for positioning one or more components of robotic system 100 relative to the group of surgical accessory elements 216. As shown, the display includes an image 702 of scene 214 that may be captured by sensors 204. The display further includes one or more augmentations in the form virtual overlays 704 (e.g., virtual overlays 704-1 to 704-3) associated with the group of surgical accessory elements 216 on image 702 of scene 214.

The one or more virtual overlays 704 may be configured to assist with positioning robotic system 100 relative to the group of surgical accessory elements 216. For example, virtual overlay 704-1 includes a focal point representative of a location of manipulator assembly 102. Virtual overlay 704-2 includes a trajectory from the focal point toward the group of surgical accessory elements 216 representative of a proposed direction of movement for manipulator assembly 102. In some implementations, the trajectory may be generated around an obstacle (e.g., other components in scene 214) between robotic system 100 and the group of surgical accessory elements 216 (e.g., to avoid a collision between robotic system 100 and the obstacle). Virtual overlay 704-3 includes a highlight of one or more surgical accessory elements 216 that may be representative of a location of the group of surgical accessory elements 216. These virtual overlays 704 are merely illustrative. Any other suitable number and/or types of virtual overlays 704 may be used with reference to the group of surgical accessory elements 216, manipulator assembly 102, and/or other components of robotic system to assist with positioning robotic system 100. In some other implementations, virtual overlays 704 may include text instructions for directing movement of one or more components of robotic system 100 and/or a display of a distance (e.g., distance D) of the group of surgical accessory elements 216 relative to robotic system 100.

User interface 206 may further comprise one or more user inputs 706, as shown in FIG. 7. The one or more inputs 706 may be configured to receive input from a user 110 such as to direct movement of one or more components of robotic system 100 for positioning robotic system 100 relative to the group of surgical accessory elements 216 and/or otherwise control operation of setup system 202. While the illustrated implementation shows a user input 706 as a button, still other suitable types of user inputs 706 may be used (e.g., a keypad, a touchscreen, a joystick, etc.) User interface 206 further includes, in the illustrated example, a handle 708 or (other suitable device) that may be configured to allow a user 110 to physically move (e.g., push or pull) manipulator assembly 102 and/or other component of robotic system 100. Still other suitable methods for positioning robotic system 100 may be used.

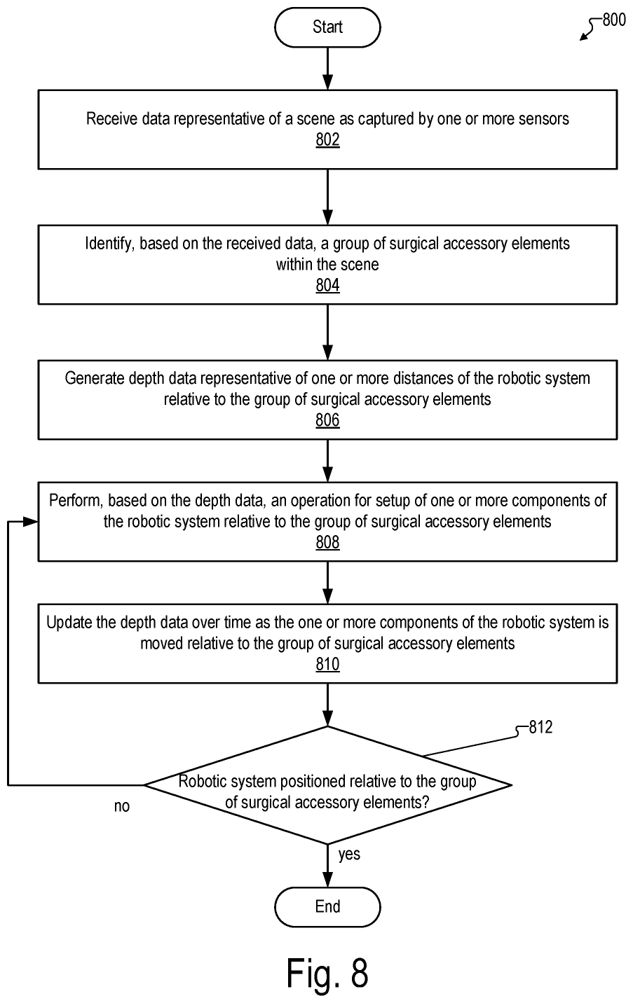

FIG. 8 shows another illustrative method 800 that may be performed by setup system 202. While FIG. 8 illustrates exemplary operations according to one embodiment, other embodiments may omit, add to, reorder, and/or modify any of the operations shown in FIG. 8. Moreover, each of the operations depicted in FIG. 8 may be performed in any of the ways described herein.

As shown, setup system 202, may at operation 802, receive data representative of scene 214 as captured by one or more sensors 204. Setup system 202 may, at operation 804, identify, based on the received data, the group of surgical accessory elements 216 within scene 214. For example, setup system 202 may identify the group of surgical accessory elements 216 by detecting one or more markers of the group of surgical accessory elements 216 and/or by machine learning algorithms. In some implementations, setup system 202 may identify one or more surgical accessory elements 216 relative to other surgical accessory elements 216 (e.g., based on different markers of the surgical accessory elements 216). In some implementations, the number of markers identified by setup system 202 may indicate the number of surgical accessory elements within the group of surgical accessory elements 216.

Setup system 202 may determine information representative of one or more positions of the identified group of surgical accessory elements 216 within scene 214. In some implementations, the one or more positions of surgical accessory elements 216 may include one or more 3D positions of surgical accessory elements 216. For instance, setup system 202, may at operation 806, generate depth data representative of one or more distances of the robotic system relative to the group of surgical accessory elements 216. The depth data may be generated based on light reflected from a marker of the group of surgical accessory elements 216 and/or by processing stereoscopic images of the group of surgical accessory elements 216. The one or more distances may include a distance from a component of robotic system 100 to a target location of surgical accessory elements 216 (e.g., an individual surgical accessory element 216, a centroid of the surgical accessory elements 216, and/or another location associated with the surgical accessory elements 216).

In some implementations, the depth data generated based on the group of surgical accessory elements 216 may provide a directional context within scene 214. For example, the depth data associated with the group of surgical accessory elements 216 may be indicative of a directional alignment of the group of surgical accessory elements 216 relative to each other such that setup system 202 may detect a directional alignment (e.g., a frontal axis, a sagittal axis, and/or a vertical axis) of the patient within scene 214.

In some implementations, setup system 202 may be configured to determine a quality value associated with the depth data. For example, setup system 202 may determine whether a quality of the data representative of scene 214 received by setup system 202 is sufficient for generating the depth data. To illustrate, setup system 202 may determine the quality of the data representative of scene 214 based on characteristics (e.g., an amount of noise, an amount of incomplete data, errors, etc.) of the data. In some implementations, setup system 202 may detect whether the number of surgical accessory elements identified within the group of surgical accessory elements 216 corresponds to a desired number of surgical accessory elements (e.g., based on the type of medical procedure). Additionally or alternatively, setup system 202 may be configured to determine whether a quality of the depth data is sufficient for determining the one or more distances associated with the group of surgical accessory elements 216 (e.g., using clustering algorithms). In instances where the quality of the data representative of scene 214 and/or the depth data is insufficient (e.g., below a quality threshold), setup system 202 may provide a notification (e.g., by display device 218) of the insufficient quality. Alternatively, in instances where the quality of the data representative of scene 214 and/or the depth data is sufficient (e.g., at or above a quality threshold), setup system 202 may proceed with using the depth data.

For example, setup system 202 may, at operation 808, perform, based on the depth data, an operation for setup of one or more components of robotic system 100 relative to the group of surgical accessory elements 216. For example, setup system 202 may position robotic system 100 relative to the group of surgical accessory elements 216 and/or provide instructions for positioning robotic system 100 relative to the group of surgical accessory elements 216. In some implementations, setup system 202 may position robotic system 100 relative to the group of surgical accessory elements 216 based on the directional context within scene 214 indicated by the depth data associated with the group of surgical accessory elements 216. Additionally or alternatively, setup system 202 may position robotic system 100 relative to the group of surgical accessory elements 216 based on the number of surgical accessory elements identified within the group of surgical accessory elements 216. To illustrate, setup system 202 may determine a number of manipulator arms 112 of robotic system 100 to deploy and/or stow based on the number surgical accessory elements identified within the group of surgical accessory elements 216.

Setup system 202 may, in some instances, position robotic system 100 relative to the group of surgical accessory elements 216 based on receiving user input associated with the group of surgical accessory elements 216. For example, a user may interact with one or more user inputs (e.g., user inputs 706) to designate the type of medical procedure that may correspond to a desired configuration of the group of surgical accessory elements 216 such that setup system 202 may position robotic system 100 relative to the group of surgical accessory elements 216 to correspond to the desired configuration for performing the designated type of medical procedure. Additionally or alternatively, setup system 202 may identify one or more of the surgical accessory elements (e.g., as an endoscope port, an assist port, an accessory, etc.) within the group of surgical accessory elements 216 for setup of robotic system 100 based on user input designating the one or more surgical accessory elements. In some implementations, the identifying the one or more surgical accessory elements may allow robotic system 100 to provide downstream guidance (e.g., during the medical procedure).

In some implementations, setup system 202 may be configured to perform the operation for setup using a closed-loop configuration. For example, as one or more components of robotic system 100 is moved or positioned relative to the group of surgical accessory elements 216, setup system 202 may, at operation 810, update the depth data over time (e.g., using a SLAM algorithm, processing images captured by sensors 204, etc.). In some instances, the group of surgical accessory elements 216 may become obstructed from sensors 204 while robotic system 100 is moved or positioned such that setup system 202 may base the movement or position of robotic system 100 on previously generated depth data (e.g., a last-known distance from a component of robotic system 100 to a target location of surgical accessory elements 216). Additionally or alternatively, the group of surgical accessory elements 216 may be moved or positioned relative to robotic system 100 (e.g., based on movement of operating room table 118) such that setup system 202 may detect whether the surgical accessory elements within the group of surgical accessory elements 216 are moved or positioned together and/or individually.

Setup system 202 may, at operation 812, determine whether robotic system 100 is sufficiently positioned relative to the group of surgical accessory elements 216. For example, setup system 202 may determine whether a desired component of robotic system 100 is aligned with a target location of the group of surgical accessory elements 216 and/or an individual surgical accessory element 216. If robotic system 100 is not sufficiently positioned (no, at operation 812), setup system 202 may continue to perform an operation for setup of one or more components of robotic system 100 (e.g., direct movement of robotic system 100). If robotic system is sufficiently positioned (yes, at operation 812), setup system 202 may abstain from further directing movement of robotic system 100. In some implementations, setup system 202 may cause movement of robotic system 100 until one or more components of robotic system is proximate to the group of surgical accessory elements 216. A user 110 may then manually move robotic system 100 to align the one or more components of robotic system 100 with the group of surgical accessory elements 216 such as to complete setup of one or more components of robotic system 100. In some implementations, setup system 202 may provide a notification (e.g., by display device 218) that setup of one or more components of robotic system 100 relative to the group of surgical accessory elements 216 is complete. Additionally or alternatively, setup system 202 may be configured to perform the operation for setup using an open-loop configuration (e.g., setup system 202 may not be configured to update the depth data over time).

While certain examples described herein are directed to capture and use of 3D data to represent a scene 214 and 3D positions of surgical accessory elements 216 within scene 214, in other examples 2D data may be captured and used to represent scene 214 and 2D positions of surgical accessory elements 216 within scene 214. For example, one or more sensors 204 may capture one or more 2D images of scene 214. Using the one or more 2D images, setup system 202 may identify a group of surgical accessory elements 216 within scene 214 and perform, based on information representing 2D positions of the group of surgical accessory elements 216 within scene 214, an operation for setup of one or more components of robotic system 100 relative to the group of surgical accessory elements 216.

Performing an operation of setup of one or more components of a robotic system 100 based on a group of surgical accessory elements 216 may provide certain advantages over performing an operation for setup based on a single surgical accessory element. For example, the group of surgical accessory elements 216 may allow a group of components of robotic system 100 (e.g., manipulator arms 112) to be collectively mapped to access the group of surgical accessory elements 216. For example, manipulator arms 112 may be collectively moved into alignment with a single target location or a group of target locations associated with the group of surgical accessory elements 216. For instance, the group of surgical accessory elements 216 may be used to determine a target location that is optimal for setup of one or more components of robotic system 100 relative to the group of surgical accessory elements 216 considered as a whole (e.g., relative to a centroid or other central target location). The group of surgical accessory elements 216 may further identify a number of manipulator arms 112 that may be deployed (e.g., for performing a medical procedure).

The group of surgical accessory elements 216 may further be used to determine a select surgical accessory element that is optimal to use as a target for setup of the one or more components of robotic system 100. To illustrate, a select manipulator arm 112 coupled or to be coupled with an endoscope may be aligned with a select surgical accessory element 216 identified as an endoscope port. Additionally or alternatively, two or more surgical accessory elements of the group of surgical accessory elements 216 may be used to determine a target location for the one or more components of robotic system 100 and/or a directional context within scene 214. For example, the combination of operating room table 118 and a cannula may be used to determine a target location for manipulator assembly 102. As another example, the combination of multiple cannulas (or other access elements) may be used to determine a target location for manipulator assembly 102. In some implementations, the group of surgical accessory elements 216 may be used to determine a quality associated with the positioning of the one or more components of robotic system 100.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

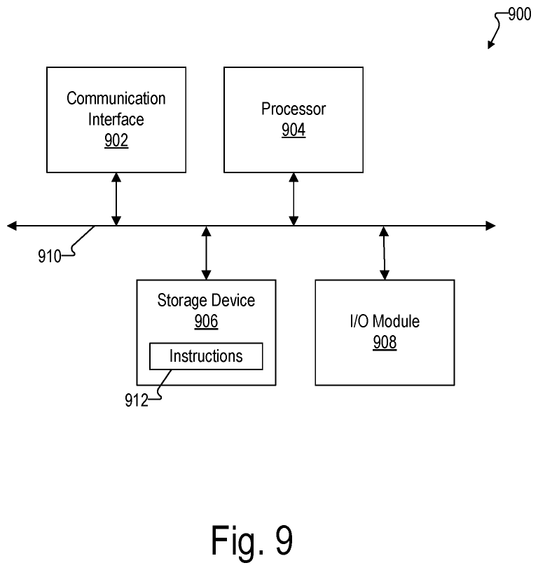

FIG. 9 shows an illustrative computing device 900 that may be specifically configured to perform one or more of the processes described herein. Any of the systems, computing devices, and/or other components described herein may be implemented by computing device 900.

As shown in FIG. 9, computing device 900 may include a communication interface 902, a processor 904, a storage device 906, and an input/output (“I/O”) module 908 communicatively connected one to another via a communication infrastructure 910. While an illustrative computing device 900 is shown in FIG. 9, the components illustrated in FIG. 9 are not intended to be limiting. Additional or alternative components may be used in other embodiments. Components of computing device 900 shown in FIG. 9 will now be described in additional detail.

Communication interface 902 may be configured to communicate with one or more computing devices. Examples of communication interface 902 include, without limitation, a wired network interface (such as a network interface card), a wireless network interface (such as a wireless network interface card), a modem, an audio/video connection, and any other suitable interface.

Processor 904 generally represents any type or form of processing unit capable of processing data and/or interpreting, executing, and/or directing execution of one or more of the instructions, processes, and/or operations described herein. Processor 904 may perform operations by executing computer-executable instructions 912 (e.g., an application, software, code, and/or other executable data instance) stored in storage device 906.

Storage device 906 may include one or more data storage media, devices, or configurations and may employ any type, form, and combination of data storage media and/or device. For example, storage device 906 may include, but is not limited to, any combination of the non-volatile media and/or volatile media described herein. Electronic data, including data described herein, may be temporarily and/or permanently stored in storage device 906. For example, data representative of computer-executable instructions 912 configured to direct processor 904 to perform any of the operations described herein may be stored within storage device 906. In some examples, data may be arranged in one or more databases residing within storage device 906.

I/O module 908 may include one or more I/O modules configured to receive user input and provide user output. I/O module 908 may include any hardware, firmware, software, or combination thereof supportive of input and output capabilities. For example, I/O module 908 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 908 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
a processor communicatively coupled to the memory and configured to execute the instructions to:
identify, based on data representative of a scene as captured by one or more sensors, a group of surgical accessory elements within the scene, wherein each surgical accessory element included in the group of surgical accessory elements is unattached to the robotic system and includes one or more markers for use in the identifying the group of surgical accessory elements within the scene; and
perform, based on information representative of one or more positions of the group of surgical accessory elements within the scene, an operation for setup of one or more components of a robotic system relative to the group of surgical accessory elements.

2. (canceled)

3. The system of claim 1, wherein
the identifying the group of surgical accessory elements includes detecting the one or more markers in the scene.

4. The system of claim 3, wherein each of the one or more markers include a retroreflective material.

5. The system of claim 1, wherein the group of surgical accessory elements includes:
a first surgical accessory element comprising a first retroreflective marker; and
a second surgical accessory element comprising a second retroreflective marker different from the first retroreflective marker.

6. (canceled)

7. The system of claim 1, wherein the information representative of one or more positions of the group of surgical accessory elements includes depth data representative of one or more distances of the group of surgical accessory elements relative to one or more components of the robotic system.

8. The system of claim 7, wherein the processor is further configured to execute the instructions to update the depth data over time as one or more components of the robotic system is moved relative to the group of surgical accessory elements.

9. The system of claim 7, wherein the processor is further configured to determine a quality value associated with the depth data and provide, for display by a display device, a notification when the quality value is below a threshold.

10. The system of claim 1, wherein the identifying the group of surgical accessory elements includes identifying a pose of each surgical accessory element of the group of surgical accessory elements within the scene.

11. The system of claim 1, wherein the operation for setup of one or more components of the robotic system includes positioning one or more components of the robotic system relative to the group of surgical accessory elements.

12. The system of claim 11, wherein the positioning of one or more components of the robotic system includes aligning one or more components of the robotic system with the group of surgical accessory elements.

13. The system of claim 11, wherein the positioning of one or more components of the robotic system includes aligning one or more components of the robotic system with an individual surgical accessory element of the group of surgical accessory elements.

14. The system of claim 11, wherein the positioning of one or more components of the robotic system includes directing, based on kinematic data of the robotic system, one or more components of the robotic system to move relative to the group of surgical accessory elements.

15. The system of claim 11, wherein the positioning of one or more components of the robotic system is based on a directional alignment of the surgical accessory elements within the group of surgical accessory elements relative to each other.

16. The system of claim 11, wherein the positioning of one or more components of the robotic system is based on a number of the surgical accessory elements within the group of surgical accessory elements.

17. The system of claim 11, wherein the positioning of one or more components of the robotic system is based on detecting a user input associated with the group of surgical accessory elements.

18. The system of claim 11, wherein the positioning of one or more components of the robotic system further includes providing, for display by a display device, a notification when one or more components of the robotic system are positioned relative to the group of surgical accessory elements.

19. The system of claim 1, wherein the operation for setup of one or more components of the robotic system includes providing instructions to a user interface for positioning one or more components of the robotic system relative to the group of surgical accessory elements.

20. The system of claim 1, wherein the processor is further configured to execute the instructions to display the scene on a display device, wherein the display of the scene includes one or more virtual overlays associated with the group of surgical accessory elements on the scene, the one or more virtual overlays configured to assist with positioning the robotic system relative to the group of surgical accessory elements.

21. (canceled)

22. A system comprising:
a robotic system;
one or more sensors configured to capture a scene; and
a control system communicatively coupled with the robotic system and the one or more sensors, wherein the control system is configured to:
identify, based on data representative of the scene as captured by the one or more sensors, a group of surgical accessory elements within the scene, wherein each surgical accessory element included in the group of surgical accessory elements is unattached to the robotic system and includes a marker for use in the identifying the group of surgical accessory elements within the scene; and
perform, based on information representative of one or more positions of the group of surgical accessory elements within the scene, an operation for setup of one or more components of the robotic system relative to the group of surgical accessory elements.

23. A method comprising:
identifying, by a setup system and based on data representative of a scene as captured by one or more sensors, a group of surgical accessory elements within the scene, wherein each surgical accessory element included in the group of surgical accessory elements is unattached to the robotic system and includes a marker for use in the identifying the group of surgical accessory elements within the scene; and
performing, by a setup system and based on information representative of one or more positions of the group of surgical accessory elements within the scene, an operation for setup of one or more components of a robotic system relative to the group of surgical accessory elements.

24.-42. (canceled)
